(19) United States
(12) Patent Application Publication
(10) Pub. No.: US 2026/0284434 A1
(43) Pub. Date: Sep. 24, 2026

(54) DYNAMIC ADJUSTMENT OF RADIOTHERAPY GRAPHICAL USER INTERFACES

(71) Applicant: VARIAN MEDICAL SYSTEMS, INC., Palo Alto, CA (US)

(72) Inventors: Brian SPATOLA, Huntington Beach, CA (US); Filip VOJAN, Redwood City, CA (US)

(73) Assignee: VARIAN MEDICAL SYSTEMS, INC., Palo Alto, CA (US)

(21) Appl. No.: 19/087,296

(22) Filed: Mar. 21, 2025

(51) Int. Cl. A61N 5/10; G06F 3/04817; G06F 3/04847

(52) U.S. Cl. CPC A61N 5/1048; A61N 5/1081; G06F 3/04817; G06F 3/04847; A61N 2005/1074

(57) ABSTRACT

A display screen on a gantry of a radiotherapy machine presents a GUI having a set of icons to assist an operator with guidance for setup and adjustments. During rotation of the gantry, the set of icons are dynamically adjusted on a display screen. During the dynamic adjustment, the set of icons are relocated on the display screen such that they maintain an orientation relative to a reference (e.g., a couch within the radiotherapy system) despite the rotation of the display screen. In some implementations, the dynamic adjustment includes removing the set of icons from a first position and displaying them at a second position during rotation of the gantry to avoid obfuscating the set of icons behind a component of the radiotherapy system. As a result, the operator may continue to interact with the GUI in an efficient manner even though the display screen may be rotated from its original and upright position.

### TECHNICAL FIELD

This application relates generally to graphical user interfaces and software for radiotherapy treatments and machines.

### BACKGROUND

Radiation therapy, which utilizes ionizing radiation, is a localized treatment targeting specific tissues, such as cancerous tumors. Ideally, this therapy focuses on the planning target volume (PTV) or target organ, sparing surrounding healthy tissue from excessive radiation doses to minimize damage. To ensure the prescribed dose is accurately delivered to the PTV, the patient must be precisely positioned relative to the linear accelerator, typically using a movable treatment couch on a turntable assembly. Implementing radiation therapy is a complex process involving specific guidelines, protocols, and instructions followed by various medical professionals, including clinicians, technicians, device manufacturers, and treating physicians. Due to the hazards associated with radiation, it is crucial that all instructions are meticulously followed.

Conventional radiation therapy systems are often complex to operate, requiring extensive training for efficient workflow execution. Tasks such as patient positioning, system calibration, and machine adjustments involve numerous interactions with both the patient and the radiotherapy machine. The data needed for these setups is often voluminous and complex. However, these conventional methods can be inefficient, ineffective, or even dangerous.

Conventional radiotherapy systems do not efficiently display the data needed for patient and machine setup. In some conventional systems and methods, the necessary information for an operator (e.g., radiotherapy technician or any other medical professional) is not presented within the treatment room or in a manner that is intuitive and user-friendly, interrupting the adjustment process and proving to be ineffective and time-consuming. Some other conventional systems and methods provide all the information via static and complex graphical user interfaces (GUIs). Unless an operator is highly experienced with a particular system, this can slow down the setup process and degrade the patient's experience.

### SUMMARY

Given the complexities involved and discussed herein, there is a need for an intuitive and workflow-oriented graphical user interface (GUI) specific to radiotherapy treatments that efficiently displays treatment setup data, such as machine parameters, patient alignment data, and necessary accessories. As described herein, a display screen on a gantry of a radiotherapy machine presents a GUI having a set of icons to assist an operator with guidance for setup and adjustments. During rotation of the gantry, the set of icons are dynamically adjusted on a display screen. During the dynamic adjustment, the set of icons are relocated on the display screen such that they maintain an orientation relative to a reference (e.g., a couch within the radiotherapy system) despite the rotation of the display screen. In some implementations, the dynamic adjustment includes removing the set of icons from a first position and displaying them at a second position during rotation of the gantry to avoid obfuscating the set of icons behind a component of the radiotherapy system. As a result, the operator may continue to interact with the GUI in an efficient manner even though the display screen may be rotated from its original and upright position.

In one embodiment, the techniques described herein relate to a computer-readable with instructions stored therein that when executed by one or more processors, cause the one or more processors to: present on a display screen located on a gantry of a radiotherapy machine, a set of icons corresponding to one or more attributes of a radiotherapy treatment implemented via the radiotherapy machine; receive a first indication of a movement of the gantry of the radiotherapy machine; and upon receiving the first indication of the movement of the gantry of the radiotherapy machine, relocate a subset of the set of icons on the display screen, such that the subset of the set of icons appears to be not rotating relative to a movement of the gantry of the radiotherapy machine.

The instructions may cause the one or more processors, upon receiving the first indication of the movement of the gantry of the radiotherapy machine, relocate a second subset of the set of icons on the display screen, such that the second subset of the set of icons appear to be rotating consistent with the movement of the gantry.

The instructions may cause the one or more processors to: remove the second subset of the set of icons from a first position when the gantry has rotated beyond a first defined position; and display at a second position the second subset of the set of icons previously removed when the gantry has rotated beyond a second defined position.

The second subset of the set of icons may display an identifier of a patient being treated.

The instructions may cause the one or more processors to: disable the display screen when the gantry is at a defined position.

The instructions may cause the one or more processors to: obfuscate at least one icon within the set of icons when the gantry reaches a defined position.

Relocating of the second subset of the set of icons may be consistent with a movement parameter of the movement of the gantry of the radiotherapy machine.

The instructions may cause the one or more processors to: receive a second indication of the movement parameter of the gantry of the radiotherapy machine; determine an icon movement parameter for the second subset of the set of icons, wherein the icon movement parameter corresponds to the movement parameter of the gantry; and upon receiving the first indication of the movement of the gantry of the radiotherapy machine, relocate the second subset of the set of icons on the display screen in accordance with the icon movement parameter, such that the second subset of the set of icons appear to be rotating with the movement of the gantry.

The movement parameter of the gantry of the radiotherapy machine may include at least one of a speed of rotation of the movement of the gantry, a direction of rotation of the movement of the gantry, an acceleration of the movement of the gantry, or an angle of rotation of the movement of the gantry.

Relocating the second subset of the set of icons on the display screen, such that the second subset of the set of icons appear to be rotating consistent with the movement of the gantry may further include: adjusting the second subset of the set of icons with respect to the display screen at a first angle of rotation opposite of a second angle of rotation of the movement of the gantry.

The instructions may cause the one or more processors to: maintain a location of the second subset of the set of icons relative to the display screen such that the second subset of the set of icons rotate about a datum point on the display screen and wherein the second subset of the set of icons are further adjusted at the first angle of rotation about a secondary datum point.

In another embodiment, the techniques described herein relate to a system including: a radiotherapy machine including a gantry with a display screen located thereon; one or more processors; and a computer-readable, non-transitory medium with instructions stored therein that when executed by the one or more processors, cause the one or more processors to: present on the display screen located on the gantry of the radiotherapy machine, a set of icons corresponding to one or more attributes of a radiotherapy treatment implemented via the radiotherapy machine; receive a first indication of a movement of the gantry of the radiotherapy machine; and upon receiving the first indication of the movement of the gantry of the radiotherapy machine, relocate a subset of the set of icons on the display screen, such that the subset of the set of icons appear to be not rotating relative to the movement of the gantry of the radiotherapy machine.

The instructions may further cause the one or more processors to, upon receiving the first indication of the movement of the gantry of the radiotherapy machine, relocate a second subset of the set of icons on the display screen, such that the second subset of the set of icons appear to be rotating consistent with the movement of the gantry.

The instructions may further cause the one or more processors to: remove the second subset of the set of icons from a first position when the gantry has rotated beyond a first defined position; and display at a second position the second subset of the set of icons previously removed when the gantry has rotated beyond a second defined position.

Relocating of the second subset of the set of icons may correspond with a movement parameter of the movement of the gantry of the radiotherapy machine.

The instructions may further cause the one or more processors to: receive a second indication of the movement parameter of the gantry of the radiotherapy machine; determine an icon movement parameter for the second subset of the set of icons, wherein the icon movement parameter corresponds to the movement parameter of the gantry; and upon receiving the first indication of the movement of the gantry of the radiotherapy machine, relocate the second subset of the set of icons on the display screen in accordance with the icon movement parameter, such that the second subset of the set of icons appear to be rotating with the movement of the gantry while maintaining a first orientation.

The instructions may further cause the one or more processors to adjust a first orientation of the second subset of the set of icons with respect to the display screen at a first angle of rotation opposite of a second angle of rotation of the movement of the gantry such that a second orientation of the second subset of the set of icons is maintained when relocating the second subset of the set of icons on the display screen, such that the second subset of the set of icons appears to be rotating consistent with the movement of the gantry.

The instructions further cause the one or more processors to maintain a location of the second subset of the set of icons relative to the display screen such that the second subset of the set of icons rotate about a datum point on the display screen.

In yet another embodiment, the techniques described herein relate to a method including: presenting, by at least one processor on a display screen located on a gantry of a radiotherapy machine, a set of icons corresponding to one or more attributes of a radiotherapy treatment implemented via the radiotherapy machine; receiving, by the at least one processor, a first indication of a movement of the gantry of the radiotherapy machine; and upon receiving the first indication of the movement of the gantry of the radiotherapy machine, relocating, by the at least one processor, a subset of the set of icons on the display screen, such that the subset of the set of icons appear to be not rotating relative to the movement of the gantry of the radiotherapy machine.

The method may further include: upon receiving the first indication of the movement of the gantry of the radiotherapy machine, relocating, by the at least one processor, a second subset of the set of icons on the display screen, such that the second subset of the set of icons appear to be rotating consistent with the movement of the gantry.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates components of a workflow-oriented radiotherapy system, according to an embodiment.

FIG. 2A illustrates a radiotherapy machine in a default, upright orientation, according to an embodiment.

FIG. 2B illustrates a radiotherapy machine in a rotated orientation, according to an embodiment.

FIG. 3A illustrates a radiotherapy machine in a default, upright orientation and displaying a set of icons, according to an embodiment.

FIG. 3B illustrates a radiotherapy machine in a rotated orientation and displaying a set of icons, according to an embodiment.

FIG. 3C illustrates a radiotherapy machine in a rotated orientation and displaying a set of icons, according to an embodiment.

FIG. 3D illustrates a radiotherapy machine in a rotated orientation and displaying a set of icons, according to an embodiment.

FIG. 4A illustrates a radiotherapy machine in a default, upright orientation and displaying a set of icons, according to an embodiment.

FIG. 4B illustrates a radiotherapy machine in a rotated orientation and displaying a set of icons, according to an embodiment.

FIG. 4C illustrates a radiotherapy machine in a rotated orientation and displaying a set of icons, according to an embodiment.

FIG. 4D illustrates a radiotherapy machine in a rotated orientation and displaying a set of icons, according to an embodiment.

FIG. 5 is a flow diagram of a method for dynamically adjusting a position and orientation of a set of icons on a radiotherapy machine during rotation of the radiotherapy machine, according to an embodiment.

### DETAILED DESCRIPTION

Reference will now be made to some embodiments illustrated in the drawings, and specific language will be used here to describe the same. It will nevertheless be understood that no limitation of the scope of the embodiments of the methods and systems described herein is thereby intended. Alterations and further modifications of the features illustrated here, and additional applications of the principles of the embodiments of the methods and systems described herein, as illustrated here, which would occur to a person skilled in the relevant art and having possession of this disclosure, are to be considered within the scope of the embodiments, methods, and/or systems described herein.

Certain radiotherapy machines used during administration of radiotherapy treatments include screens mounted to the radiotherapy machine to aid in the administration of the radiotherapy treatment. These screens may be mounted to a gantry of the machine, which is able to rotate around a patient to be treated during set up and delivery of the radiotherapy. The screen displays a GUI having multiple icons that provide details related to the radiotherapy process, such as setup parameters/adjustments, real-time positioning information, and/or patient information. As the gantry of the radiotherapy machine rotates, the screen may counter-rotate to allow for a consistent orientation of the GUI when the gantry and the screen of the gantry have changed position.

To maintain legibility of the icons during rotation of the gantry, positions of the icons within both categories of icons are dynamically adjusted on the GUI during the rotation of the gantry. The first category of icons may be located at a periphery of the display screen. The icons in the first category of icons are dynamically adjusted during gantry rotation so that they appear to rotate as the gantry rotates but maintains an upright orientation. The icons in the second category of icons are also dynamically adjusted during gantry rotation so that they appear to be stationary, irrespective of the gantry's rotation.

### System Architecture

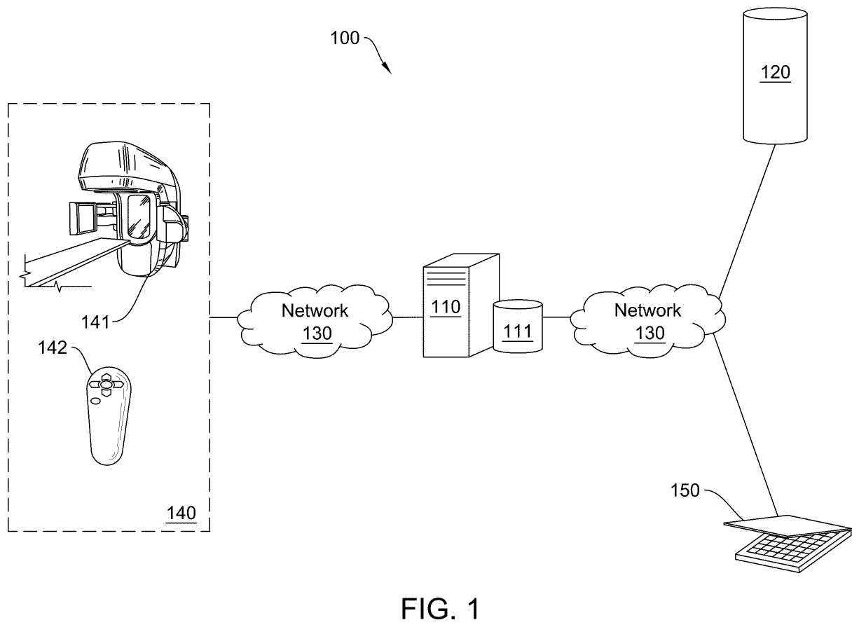

The methods described herein can be implemented using various computing devices/features described in FIG. 1. Therefore, FIG. 1 describes a non-limiting example of a computer environment where a server can perform the processes/methods described herein, such as retrieving, processing, and presenting radiotherapy treatment data, and presenting data for a graphical user interface (GUI) having workflow-oriented pages (or instances) on various displays screens.

FIG. 1 illustrates various components of a system 100 for presenting various pages related to operation of a radiotherapy treatment, in accordance with an embodiment. The system 100 may include an analytics server 110, a medical records database 120, a radiotherapy system 140, and a system administrator computer 150 or workstation. These features may communicate with each other over a network 130. For example, the system 100 may present, via the analytics server 110, one or more pages/GUIs on the radiotherapy machine 141. In another example, the system 100 may present, via the analytics server 110, a page presenting a live feed of a patient on one or more display devices, such as the display device on a gantry of the radiotherapy machine 141.

The network 130 may include wired and/or wireless communications according to one or more standards via one or more transport mediums. Communication over the network 130 may be in accordance with various communication protocols, such as transmission control protocol and internet protocol (TCP/IP), user datagram protocol (UDP), and Institute of Electrical and Electronics Engineers (IEEE) communication protocols. The network 130 may further include wireless communications according to Bluetooth specification sets, or another standard or proprietary wireless communication protocol. The network 130 may further include communications over a cellular network, including, for example, a global system for mobile (GSM) communications, code division multiple access (CDMA), and enhanced data for global evolution network (EDGE). The examples of the network 130 may include, but are not limited to, private or public local area network (LAN), wireless LAN (WLAN), metropolitan area network (MAN), wide area network (WAN), and the Internet.

The analytics server 110 may be any computing device capable of performing the actions described herein. For instance, the analytics server 110 includes a processing unit and a non-transitory machine-readable storage medium. The processing unit includes a processor with a computer-readable medium, such as a random-access memory coupled to the processor. In some embodiments, the analytics server 110 executes algorithms or computer executable program instructions, which may be executed by a single processor or multiple processors in a distributed configuration. The instructions allow the processor to implement the functionality described herein. The analytics server 110 may be configured to interact with one or more software modules of a same or a different type operating within the system 100.

Non-limiting examples of the processor may include a microprocessor, an application specific integrated circuit, and a field programmable object array, among others. The analytics server 110 may be capable of executing data processing tasks, data analysis tasks, and valuation tasks. Non-limiting examples of the analytics server 110 may include a desktop computer, a server computer, a laptop computer, a tablet computer, and the like. For simplicity, the FIG. 1 depicts a single-server computing device functioning as the analytics server 110. However, some embodiments may include a plurality of server computing devices capable of performing various tasks described herein.

Implementation of the methods described herein is not limited to the system architecture depicted in FIG. 1. In alternative embodiments, the analytics server 110 may be an embedded computing device disposed within the radiotherapy machine 141. In some embodiments, the analytics server 110 may be a plurality of computing devices operated locally and/or remotely. In various embodiments, the analytics server 110 may be operated through a cloud service (e.g., network, internet). The cloud service may be performed in accordance with various communication protocols such as TCP/IP, UDP, and IEEE communication protocols. The analytics server 110 may utilize a database, such as a local database 111, to store and/or retrieve various data described herein. For instance, the analytics server 110 may store different data corresponding to the different pages within the local database 111. The page may be displayed on a display screen associated with the radiotherapy system 140.

For instance, the analytics server 110 may display a page having a live feed of the patient before the treatment has begun or may display various pages describing how to position the patient on the couch and/or how to adjust the radiotherapy machine 141. Even though some embodiments describe the analytics server 110 displaying various pages on a display screen attached to the radiotherapy machine 141 (e.g., located on the gantry of the radiotherapy machine 141), it is expressly understood that the analytics server 110 may display different pages described herein on a display screen located anywhere within the treatment room, such as located on the wall of the treatment room or on any electronic device within the treatment room. In some configurations, the analytics server 110 may display the pages on the system administrator computer 150 or workstation.

The local database 111 may also store data corresponding to the radiotherapy machine 141 (e.g., default position of the radiotherapy machine 141, current position of the radiotherapy machine 141, such as the orientation of the bed/couch and gantry).

If the analytics server 110 receives a request from the radiotherapy system 140 to provide the current position/orientation of the radiotherapy machine 141, the analytics server 110 may query the local database 111 and may be retrieve the corresponding data. Additionally, or alternatively, the analytics server 110 may also communicate with various sensors associated with the radiotherapy system 140 to retrieve machine data and parameters. The analytics server 110 may then use the methods/systems described herein to instruct the radiotherapy machine 141 to adjust the positioning of the radiotherapy machine 141 to the default position of the radiotherapy machine 141, or another position of the radiotherapy machine, or to display one or more pages instructing a technician to adjust the radiotherapy machine 141 and/or the patient.

The local database 111 may also store data associated with different users of the radiotherapy system 140. In a non-limiting example, the user data may include the user's login (e.g., sign-in, credentials) information and authorization levels. For example, the analytics server 110 may store, using the local database 111 may store, the user's name and password. The analytics server 110 may allow the user to log in to the radiotherapy machine 141, and/or the system administrator computer 150. If the user logs-in into the system, the analytics server 110 may adjust what the user is able to view, adjust, and control based on the user's authorization level. The analytics server 110 may conceal the patient's medical information that is not relevant to the radiation treatment from the medical technician. In another example, the analytics server 110 may conceal all of the patient's medical information if a machine technician signs-in.

The analytics server 110 may also utilize one or more other databases, such as the medical records database 120, to store and/or retrieve various data described herein. The databases herein can be configured as one or more databases storing the data, and the disclosure is not intended to be limited to a particular number or location of databases. The analytics server 110 may instruct the medical records database 120 to store patient data (e.g., patient name, patient machine alignment information) associated with a patient identifier (e.g., patient's name, patient's profile image). The analytics server 110 may then instruct the medical records database 120 to populate a dataset corresponding to a patient and display the profile image of the patient. If the analytics server 110 receives a request from the radiotherapy system 140 to display the data associated with the patient identifier, the analytics server 110 may query the medical records database 120 and may retrieve the corresponding dataset. The analytics server 110 may then use the methods/systems described herein to dynamically display the patient data in accordance with the patient identifier.

The medical records database 120 may also include a radiotherapy treatment file associated with the patient identifier. As used herein, the radiotherapy treatment file refers to all data associated with a patient's radiation therapy treatment and is not limited to a particular step or information. The radiotherapy treatment file may also be retrieved from the radiotherapy system 140, the medical records database 120, and/or the system administrator computer 150. The radiotherapy treatment file may include the treatment data associated with a patient. The radiotherapy treatment file may be associated with a patient identifier and may also be updated after a patient has completed treatment. For example, after the patient has completed a treatment session, the medical records database 120 may retrieve the duration of the treatment session from the radiotherapy system 140 and update the radiotherapy file to include the duration of the treatment session. Even though aspects of the embodiments described herein discuss radiotherapy treatment as a file, the radiotherapy treatment file represents a collection of the patient's medical and treatment data, which may be stored in different files. For brevity, the present disclosure refers to the patient's data as a radiotherapy treatment file.

The radiotherapy treatment file may include data for treatment plans for one or more patients where each treatment plan is specific to a single patient. For instance, each treatment plan is uniquely created for each patient and corresponds to the patient's unique attributes (e.g., physical attributes of the patient and the patient's unique condition to be treated). In some configurations, each treatment plan for each patient may itself include multiple files.

The analytics server 110 may retrieve treatment data associated with a patient that is stored within the patient's radiotherapy treatment file(s). As described herein, the analytics server 110 may analyze the treatment data and may display various features and graphical components described herein. While the medical records database 120 may contain treatment data (radiotherapy treatment files) associated with multiple patients, the methods described herein are implemented, such that various pages and graphical features described herein are specific to the particular patient being treated.

In some configurations, the analytics server 110 may retrieve radiotherapy treatment files associated with multiple patients. The analytics server 110 may then receive a selection by an operator (e.g., technician) of a patient to be treated. As a result, the analytics server 110 identifies the radiotherapy treatment file associated with the selected patient and customizes the graphical components described herein for the selected patient.

The analytics server 110 may also retrieve and instruct the medical records database 120 to store patient data associated with the patient from the medical records database 120, the radiotherapy system 140, and/or the system administrator computer 150. For instance, the medical records database 120 may include patient data (e.g., previously populated by the analytics server 110 and/or periodically retrieved from a third-party data source). If a patient is selected, the analytics server 110 may query and retrieve patient data from the medical records database 120 and provide the retrieved patient data. For instance, the analytics server 110 may display the patient's profile image when the patient is selected, providing an opportunity to verify the correct patient was selected.

The analytics server 110 may receive treatment data associated with a patient from the medical records database 120, the treatment data may further include at least a patient identifier. The analytics server may receive radiotherapy treatment file associated with one or more patients from the medical records database 120. The radiotherapy treatment file may refer to a file having data associated with a process in which a medical team (e.g., radiation oncologists, radiation therapist, medical physicists, and/or medical dosimetrists) plan the appropriate external beam radiotherapy or internal brachytherapy treatment techniques for a patient.

The data within the radiotherapy file is not limited to the external radiation therapy as other treatments may impact the radiation therapy, such as medical oncology treatment (chemotherapy), interventional oncology treatment (e.g. cryotherapy, microwave therapy, or embolic therapy), or other non-treatment procedures, such as labs and appointments with other medical professionals. The radiotherapy treatment file may include data specific to one or more patient's radiotherapy treatment. The radiotherapy treatment file may include a patient identifier, patient's electronic health data records, medical images (e.g., CT scans, 4D CT scans, MRIs, and x-ray images), treatment-specific data (e.g., arc information or treatment type), target organ (e.g., specification and location data to identify the tumor to be eradicated), treatment plan, etc. Additional examples may include non-target organs, dosage-related calculations (e.g., radiation dose distribution within an anatomical region of the patient), and radiotherapy machine specific information (e.g., couch-gantry orientations, machine trajectory, control points, dose distributions, and/or arc information).

The analytics server 110 may use the patient identifier within the radiotherapy treatment file to identify a particular patient and retrieve additional information regarding said patient. For instance, the analytics server 110 may query the medical records database 120 to identify medical data associated with the patient. For instance, the analytics server may query data associated with the patient's anatomy, such as physical data (e.g., height, weight, and/or body mass index), and/or other health-related data (e.g., blood pressure or other data relevant to the patient receiving radiotherapy treatment). The analytics server 110 may also retrieve data associated with current and/or previous medical treatments received by the patient (e.g., prior treatment fractions, or data associated with the patient's previous surgeries).

The analytics server 110 may analyze the data received and generate additional queries accordingly. For instance, the analytics server 110 may retrieve data associated with one or more medical (or other) devices needed for the patient. The analytics server may retrieve data indicating that the patient suffers from a respiratory medical condition. As a result, the analytics server 110 may generate and transmit a query to the radiotherapy machine 141, or the system administrator computer 150 to identify whether the patient uses/needs a ventilator.

If necessary, the analytics server 110 may also analyze the patient's medical data records to identify the needed patient attributes. For instance, the analytics server 110 may query a database to identify the patient's BMI. However, because many medical records are not digitalized, the analytics server 110 may not receive the patient's BMI value using simple query techniques. As a result, the analytics server 110 may retrieve the patient's electronic health data and may execute one or more analytical protocols (e.g., natural language processing) to identify the patient's body mass index. In another example, if the analytics server 110 does not receive tumor data (e.g., end-points), the analytics server 110 may execute various image recognition protocols and identify the tumor data.

Another example of a patient attribute may include specific tumor locations. More specifically, this data may indicate the primary tumor location with respect to the patient's centerline. This data may be inputted by the treating oncologist or may be analyzed using various image recognition or segmentation methods executed on the patient's medical images.

Another example of a patient attribute may include whether the patient uses prosthesis (e.g., hip or femoral head prosthesis). This attribute may result in a change of the patient's treatment (e.g., patients with these conditions might require a special treatment).

The analytics server 110 may use various application-programming interfaces (APIs) to communicate with different features described herein. As used herein, an API refers to a computing interface that uses connector programming code to act as a software intermediary between at least two computing components/features described herein. The API may automatically and/or periodically transfer various calls, instructions, and/or requests among different features of the system 100. Using different APIs, the analytics server 110 may automatically transmit and/or receive calls and instructions.

Additionally, or alternatively, the analytics server 110 may use a content delivery network (CDN) to ensure data integrity when communicating with different features described in the system 100. As described herein, a CDN refers to a distributed delivery network of proxy servers/nodes that uses multi-layered delivery methods/systems to transmit data (e.g., Akamai). The analytics server 110 may use a CDN when communicating various calls/instructions with the network 130 and/or the local database 111.

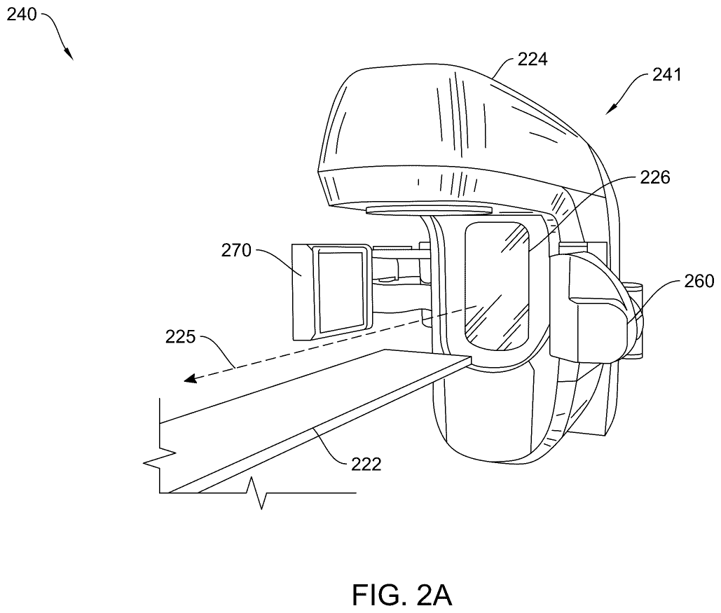

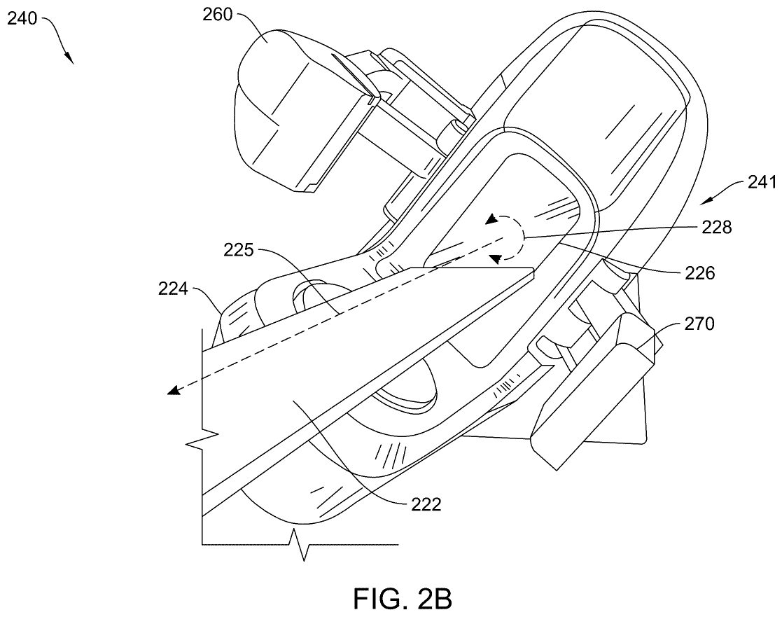

The radiotherapy machine 141 may also include a couch (shown as couch 222 in FIG. 2A) to align the patient in a designated position before the treatment begins. The designated position may be identified, calculated, and/or retrieved by the analytics server 110. For example, the analytics server 110 may retrieve patient data from the medical records database 120, analyze the data, and utilize the analyzed data to position the patient on the couch. In some embodiments, the doctor identifies how to align the patient on the couch to optimize the treatment therapy. The radiotherapy machine 141 directs radiation at specified locations of the patient resting on the couch during treatment. The specified locations may be selected by the technician operating the radiotherapy machine 141, the pendant 142, and/or the system administrator computer 150. The analytics server 110 may also specify the locations on the patient to direct the radiation.

The radiotherapy machine 141 may also include an x-ray emitter and an x-ray receiver. The x-ray emitter may be disposed on a gantry of the radiotherapy machine 141 and may be configured to provide x-ray (e.g., a penetrating form of high-energy electromagnetic radiation) waves. The x-ray emitter emits x-ray waves to the patient who is positioned on the couch. The x-ray receiver may be disposed opposed to the x-ray emitter. The x-ray waves received by the x-ray receiver generate an imprint (e.g., image) of the patient's internal anatomy. Although the example embodiment recites the use of x-ray imaging, an alternative configuration for the radiotherapy machine may include an additional or other medical imaging apparatus (e.g., fluoroscopy apparatus, MRI, etc.).

The radiotherapy machine 141 may also include a set of cameras (e.g., camera on an end of the couch, gantry camera, ceiling camera, or other cameras placed within the radiotherapy room). The analytics server 110 may retrieve a live feed of the couch (e.g. with or without the patient) from the set of cameras and provide it to a display screen disposed on the radiotherapy machine 141. The set of cameras may also directly communicate with the radiotherapy system 140.

The radiotherapy machine 141 may also include a gantry that may be in communication with the analytics server 110. If the radiotherapy machine 141 is in operation, the gantry may rotate relative to the radiotherapy machine 141 to begin the treatment of the patient. The analytics server 110 may use the live feed of the set of cameras to control and/or stop the movement of the gantry. During operation, the analytics server 110 may instruct the display screen of the radiotherapy machine 141 to display the live feed of the set of cameras and/or a designated page to the display screen of the radiotherapy machine 141. In various embodiments, the analytics server 110 may not instruct the display screen of the radiotherapy machine 141 to activate the display screen of the radiotherapy machine 141 during operation. In some embodiments, the display screen of the radiotherapy machine 141 may be a touch screen and may control the pages generated by the analytics server 110 through the display screen of the radiotherapy machine 141.

The radiotherapy machine 141 may have a default home position. Before the patient receives treatment, the radiotherapy machine 141 may be reset to a position that is ergonomically desirable for the patient. The default home position may also be optimized to receive a new patient or allow the patient to more easily get off the couch.

As discussed in greater detail herein, the radiotherapy machine 141 may also be controlled by the pendant 142. The pendant 142 may be connected to the radiotherapy machine 141 through wired or wireless communications according to, for example, Bluetooth specification sets, or another standard or proprietary wireless communication protocol. In various embodiments, the pendant 142 may be connected to the radiotherapy machine 141 through a wired connection or be integrated into the radiotherapy machine 141. The pendant 142 may also be in communication with the analytics server 110. The pendant 142 may adjust the positioning of the radiotherapy machine 141 through a selection of buttons that axially actuates the radiotherapy machine 141 (e.g., couch) towards a desired direction. The technician may also use the pendant 142 to initialize the radiotherapy machine 141, and/or to have the radiotherapy machine 141 return to its home position.

The pendant 142 may also allow the technician to input patient information, which may be then communicated to the radiotherapy machine 141, the analytics server 110, and/or the medical records database 120. The pendant 142 may be used to control one or more of the steps of the setup and treatment of the patient.

The pendant 142 may also include a touch pad (e.g., tactile sensor). The touch pad may be used to control various pages generated by the analytics server 110. For example, the user may use the touch pad of the pendant 142 to control a page, generated by the analytics server 110, to proceed through the workflow steps of the radiotherapy treatment. In some embodiments, the page may be controlled through a touch screen on the radiotherapy machine 141 or elsewhere in the treatment room.

As discussed in greater detail herein, the radiotherapy system 140 may further include accessories that assist in the treatment of the patient. The analytics server 110 may communicate with the radiotherapy system 140 to select which accessories are required. The analytics server 110 may also communicate with the medical records database 120, and/or the system administrator computer 150 to determine which accessories are required. For example, a patient may require a specific accessory for their treatment. The analytics server 110 may retrieve that the patient requires a specific accessory by accessing the medical records database 120 and communicate that information to the radiotherapy system 140. The accessories may in communication with the radiotherapy system 140. For instance, as will be described below, the analytics server may use RFID tags to scan and identify the location and/or presence of various accessories. In some embodiments, the accessories are wired or otherwise integrated into the radiotherapy machine 141.

The local database 111 associated with the analytics server 110, the medical records database 120, and the radiotherapy machine 141 are capable of storing information in various formats and/or encrypted versions. The information may include data records associated with various patient information, user preferences, a set of prompts (e.g., question, query, and inquiry), attributes associated with various pages to be generated by the analytics server 110, and the like. The medical records database 120, may have a logical construct of data files, which are stored in non-transitory machine-readable storage media, such as a hard disk or memory, controlled by software modules of a database program (e.g., structured query language (SQL)), and a database management system that executes the code modules (e.g., SQL scripts) for various data queries and management functions.

The system administrator computer 150 may represent a computing device operated by a system administrator. The system administrator computer 150 may communicate with the analytics server 110. The system administrator computer 150 may be configured to display various analytic metrics where the system administrator can monitor gantry movement, patient information, and modify various thresholds/rules described herein. The system administrator computer 150 may be configurable to display certain analytics metrics when specific thresholds/rules have been exceeded. For example, the system administrator computer 150 may be alerted if a patient has moved a specified amount during treatment. The analytics server 110 may allow the system administrator computer 150 to also control and/or override the settings of the radiotherapy machine 141, and/or the pendant 142. For instance, an administrator may revise the minimum distance threshold and/or customize any feature on the pages described herein (e.g., move features within the page, color, font, shape, size, or the order of display for one or more pages).

The analytics server 110 may configure the system administrator computer 150 to review any and/or all commands made through the radiotherapy system 140 at specified process steps. The system administrator computer 150 may then allow or reject the commands made through the radiotherapy system 140. For example, before the technician starts the patient treatment using the radiotherapy machine 141, the analytics server 110 may first prompt the system administrator computer 150 to review the radiotherapy machine 141 parameters for approval. Once approved, the technician may then control the radiotherapy machine 141.

The analytics server 110 may configure the system administrator computer 150 to review any and/or all reading and/or (over)writing of patient data to and/or from the medical records database 120. For example, before the technician may (over)write patient information collected by the radiotherapy machine 141, the analytics server 110 may first prompt the system administrator computer 150 to review the patient information before it is stored in the medical records database 120.

As discussed in greater detail herein, the analytics server 110 may generate various pages that are designed to interact with the individual operating the radiotherapy machine 141, and/or the system administrator computer 150. The analytics server 110 may then generate various interactive pages and may instruct the network 130 to incorporate the generated pages displayed on the radiotherapy machine 141.

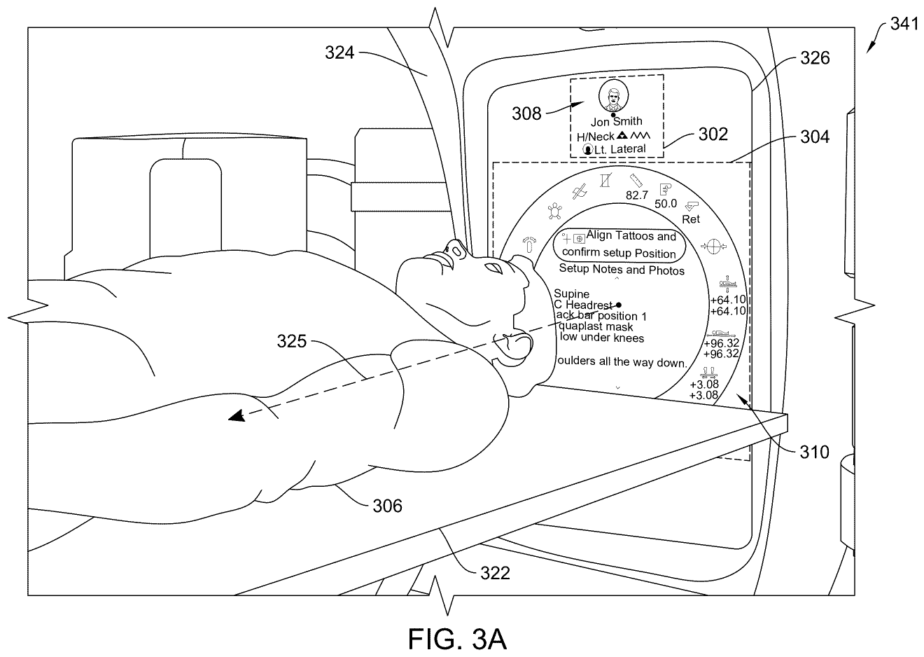

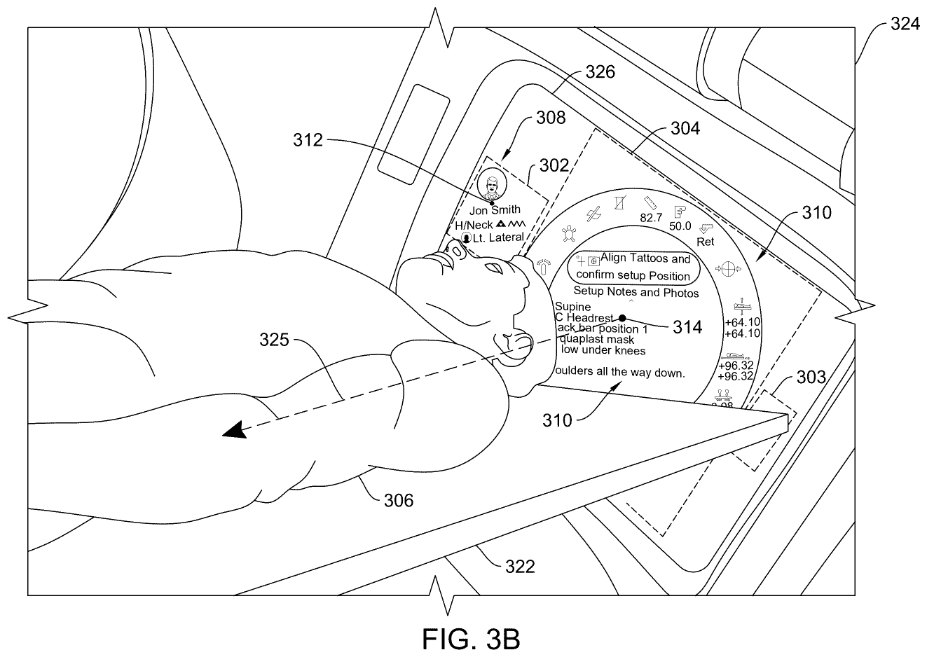

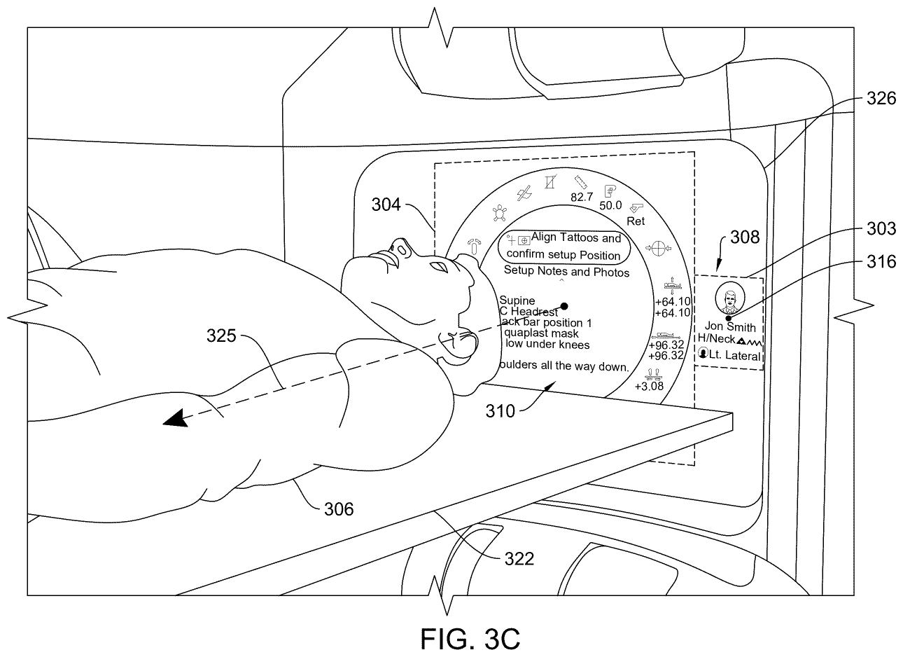

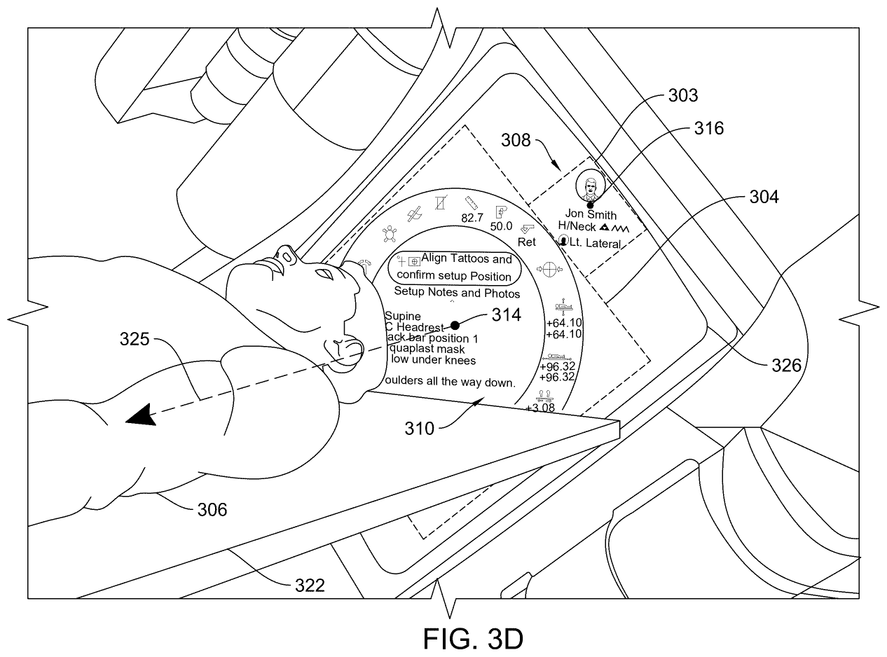

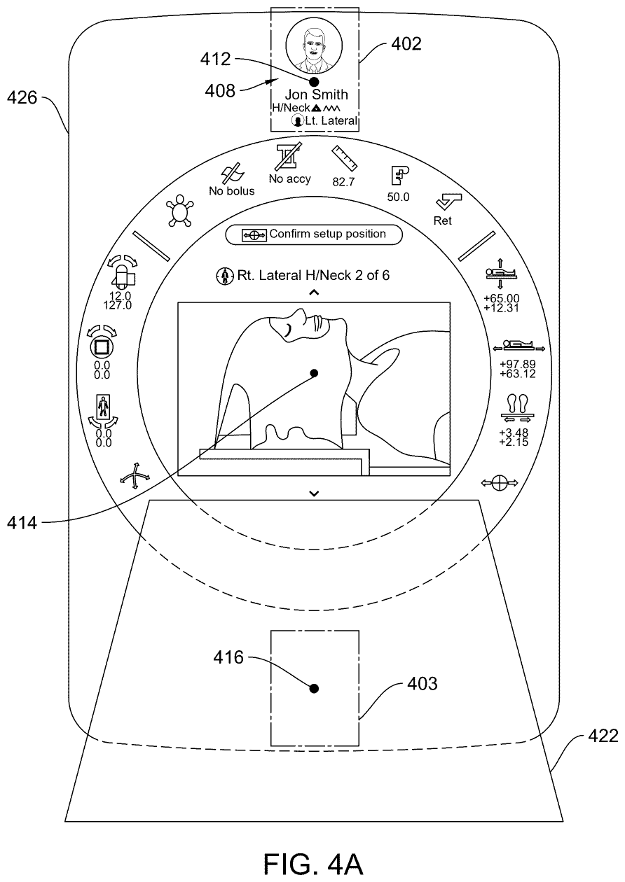

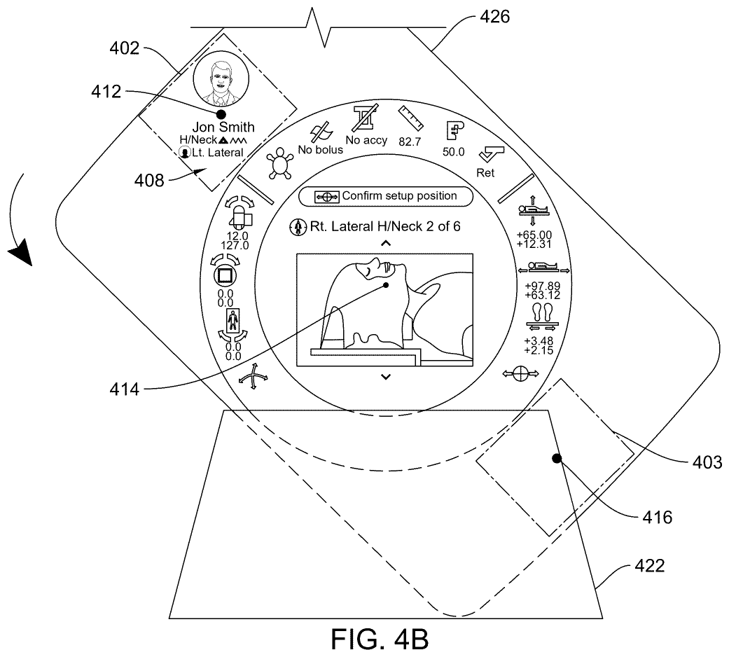

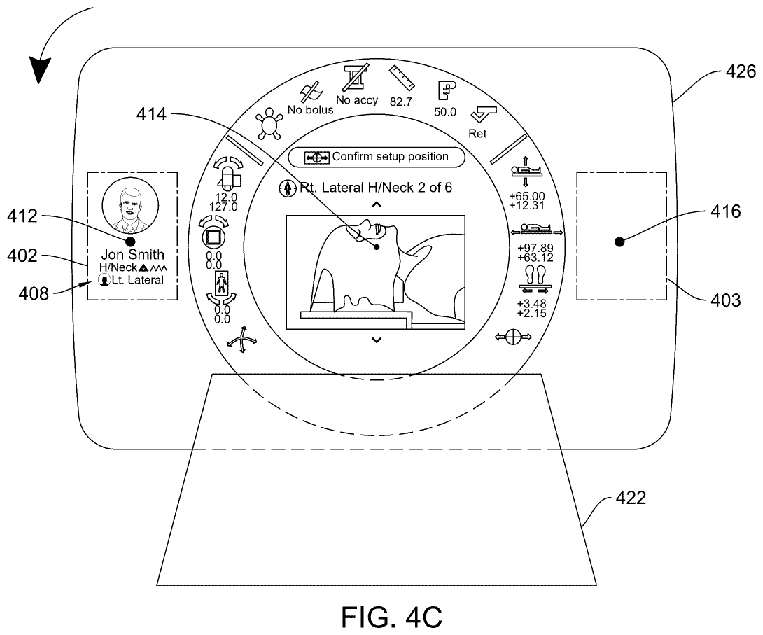

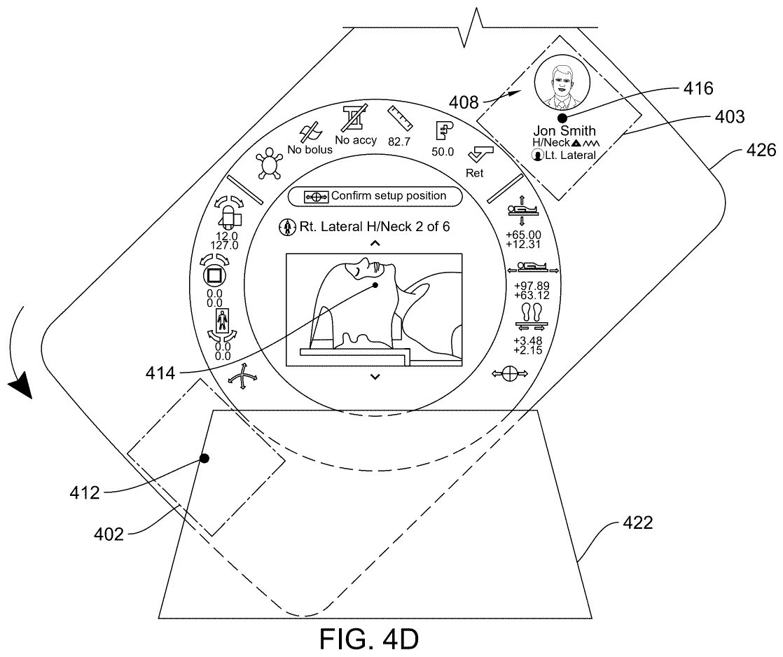

For example, in at least some embodiments of the systems and methods described herein, the analytics server 110 may present for display one or more sets of icons on a display screen coupled to the radiotherapy machine 141 to present radiotherapy information related to the radiotherapy treatment (e.g., alignment information, patient information, etc.). While the analytics server 110 is described as presenting icons on the display screen generally, it should be understood that the systems and methods described herein extend to any graphical component, such as, but not limited to, an indicator, figure, avatar, badge, symbol, marker, shape, object, emblem, arrow, direction, text, or any combination thereof. During alignment, such as during rotation of the gantry about a central axis of the radiotherapy machine 141, one or more icons within the set of icons may be hidden from a user's view (e.g., a technician, a patient, a medical practitioner) due to a location of the display screen relative to the couch or other object (as shown in FIGS. 3A-3B and 4A-4D). Additionally, or alternatively, during rotation of the gantry—and by extension, the display screen—the one or more icons displayed on the display screen may be oriented, such that they are unreadable due to being statically displayed on the display screen (e.g., the icons may be presented in an upside-down orientation when the gantry is rotated 180° from a default, upright position). In such conditions, the analytics server may dynamically adjust a position and/or rotation of the one or more icons on the display screen, such that the one or more icons are maintained in a readable orientation and position regardless of a position of the gantry of the radiotherapy machine 141. In embodiments in which the one or more icons may be obfuscated from view by a component of the radiotherapy system 140 (e.g., the couch), the analytics server 110 may dynamically adjust a position from one location on the display screen to a second position on the display screen, so as to maintain the one or more icons in a visible location during rotation of the gantry.

FIG. 2A illustrates, as described above, a radiotherapy system 240, including a radiotherapy machine 241 that includes a gantry 224 that rotates in a direction 228 (which may be clockwise or counterclockwise) about a center axis 225 of the radiotherapy machine 241. In some embodiments, the radiotherapy system 240 may be substantially similar to the radiotherapy system 140 of FIG. 1. Likewise, the radiotherapy machine 241 may be substantially similar to the radiotherapy machine 141 of FIG. 1. The radiotherapy machine 241 may further include a display screen 226 positioned on or within the gantry 224. The display screen 226 may be configured to display for view to a user (e.g., a technician, patient, and/or a medical practitioner) relevant information related to the radiotherapy procedure before, during, and/or after the radiotherapy procedure.

The radiotherapy machine 241 may also include an imager 270 and an emitter 260 to facilitate visualizing (using X-ray imaging) the location of a patient prior to and during the administration of the radiotherapy to the patient. The radiotherapy system 240 may additionally include a couch 222, upon which the patient may be placed before and during the administration of the radiotherapy. In some embodiments, the couch 222 may be translated relative to the radiotherapy machine 241 (e.g., along the center axis 225 and horizontally perpendicular to the center axis 225) and/or rotated (e.g., yaw, pitch, roll). For example, the couch 222 may rotate relative to the radiotherapy machine 241 about the center axis 225 (e.g., roll). The couch 222 may rotate relative to the radiotherapy machine 241 about an axis extending horizontally perpendicular to the center axis 225 (e.g., pitch). In some embodiments, the couch 222 may rotate relative to the radiotherapy machine 241 about an axis extending vertically perpendicular to the center axis 225 (e.g., yaw). Likewise, the radiotherapy machine 241 may be rotated axially about the center axis 225, and relative to the couch 222, before, during, and/or after the administration of the radiotherapy treatment.

For example, as illustrated in FIG. 2B, the radiotherapy machine 241 is shown in a rotated position about the center axis 225. Relative to the default position of the radiotherapy machine 241 (as shown in FIG. 2A), the radiotherapy machine 241 in FIG. 2B is shown at a position that is rotated about 135.5° counterclockwise. Because the display screen 226 is coupled to the gantry 224 of the radiotherapy machine 241, the display screen 226 is also rotated from a default, upright position (as shown in FIG. 2A) at about 135.5° counterclockwise. Though the gantry 224 and the display screen 226 are shown rotating counterclockwise, it is understood that the gantry 224 and the display screen 226 may additionally or alternatively rotate clockwise from the default position shown in FIG. 2A.

During a radiotherapy treatment procedure, as shown in FIG. 3A, a patient 306 is placed upon the couch 322 (which may be substantially similar to the couch 222 of FIG. 2A), which is positioned near the radiotherapy machine 341 (which may be substantially similar to the radiotherapy machine 241 of FIG. 2A). Before and/or during the treatment, a set of icons (e.g., graphical/textual icons) may be presented for display on the display screen 326 (which may be substantially similar to the display screen 226 of FIG. 2A). In some embodiments, the icons displayed may be divided into two separate groups of subsets. The grouping may be performed using predefined criteria (e.g., in accordance with the content or location of the icon). In some embodiments, one group (e.g., one or more icons) may appear to rotate with the gantry 324 (which may be substantially similar to the gantry 224 of FIG. 2A) and the display screen 326, while another group of icons may appear to be stationary.

In a non-limiting example shown in FIG. 3A, a subset of icons 310 of the set of icons may be presented for display in an area 304 on the display screen 326. A second subset of icons (such as the second subset of icons 308 depicted in FIG. 3A) may additionally or alternatively be presented for display at a first position 302 of the display screen 326.

The second subset of icons 308 may include personally identifiable information related to the patient 306 and/or the radiotherapy treatment. For example, the second subset of icons 308 may include a visual graphic of the patient 306 such that a medical practitioner may visually confirm an identity of the patient 306 and verify that the radiotherapy treatment being administered is the correct radiotherapy treatment. Additionally, or alternatively, the second subset of icons 308 may include text related to the patient 306 and/or personally identifiable information of the patient 306. Personally identifiable information associated with the patient 306 that may be displayed in the second subset of icons 308 on the display screen 326 may include, but is by no means limited to, a name, age, height, weight, type of treatment to be administered, emergency contact information, insurance information, a Social Security number, driver's license numbers, passport details, addresses, email addresses, phone numbers, credit card or bank account information, biometric data (such as fingerprints or facial recognition data), medical records, dates of birth, employment records, and/or educational records.

The subset of icons 310 may include information related to the setup of the radiotherapy machine 341, the couch 322, previous treatments, and/or the radiotherapy treatment more generally. For example, the subset of icons 310 may include visual and/or text graphics related to a current position of the radiotherapy machine 341 and/or the couch 322, and a desired position of the radiotherapy machine 341 and/or the couch 322. In some embodiments, the subset of icons 310 may include visual and/or text/numerical indicators that indicate a positional change to be applied to one or more of the couch 322 and/or the radiotherapy machine 341, such that the couch 222 is appropriately aligned relative to the radiotherapy machine 341 for administration of the radiotherapy treatment. In some embodiments, one or more icons within the subset of icons 310 may be dynamically updated as the radiotherapy machine 341 and/or the couch 322 are positionally adjusted.

As shown in FIG. 3A, the second subset of icons 308 of a set of icons is displayed in the first position 302 of the display screen 326. In one embodiment shown in FIG. 3A, the first position 302 is positioned at the top of the display screen 326 when the gantry 324 is in the default position. The absolute location of the first position 302 relative to the display screen 326 may be maintained during rotation of the gantry 324, as shown in FIGS. 3A-3D. In some embodiments, the second subset of icons 308 are maintained within the first position 302 of the display screen 326, regardless of position of the gantry 324 during rotation movement. Without a dynamic adjustment of the second subset of icons 308 within the first position 302 of the display screen 326, the second subset of icons 308 of the set of icons would maintain its orientation relative to the display screen 326 and be illegible when rotated. As such, in the methods and systems described herein, an analytics server (e.g., the analytics server 110 of FIG. 1) may provide instructions to the display screen 326 to dynamically relocate and/or dynamically reorient the second subset of icons 308 of the set of icons during rotation of the gantry 324, such that an orientation (e.g., the default, upright orientation) of the second subset of icons 308 of the set of icons relative to a reference object/position/location (e.g., the couch 322, gravity, etc.) is maintained during rotation, thus appearing to rotate with the gantry 324. In this way, the second subset of icons 308 is maintained in a legible orientation regardless of the position of the gantry 324. The analytics server may also provide instructions to the display screen 326 to dynamically adjust, relocate, and/or dynamically reorient the subset of icons 310 of the set of icons during rotation of the gantry 324, such that an orientation (e.g., the default, upright orientation) of the subset of icons 310 of the set of icons relative to a reference object/position/location (e.g., the couch 322, the display screen 326, gravity, etc.) is maintained during rotation, in this instance, appearing to be stationary relative to the display screen 326. In this way, the subset of icons 310 is maintained in a legible orientation regardless of the position of the gantry 324, thus providing a user of the gantry 324 with a user-friendly and intuitive display during setup of the gantry 324 in preparation of administration of the radiotherapy. This user-friendly and intuitive display decreases setup time and effort, resulting in faster radiotherapy treatment administration.

Turning now to FIGS. 3B-3D, the gantry 324 is shown rotating counterclockwise to various orientations relative to a reference, such as a reference orientation (e.g., the upright orientation shown FIG. 3A), a reference position (e.g., the upright position as shown in FIG. 3A, vertical), a reference object (e.g., the couch 322, a support such as the ground, a level, gravity), etc. In each rotated orientation of the gantry 324 in FIGS. 3B-3D, the second subset of icons 308 are maintained in an orientation and/or position relative to the reference to maintain legibility. In FIG. 3B, the gantry 324 is rotated to a position at about 45° counterclockwise from a first, upright position of the gantry 324 (e.g., as shown in FIG. 3A). The analytics server, which is communicably coupled to the radiotherapy machine 341, may receive an indication of a movement of the gantry 324, such as during movement of the gantry 324 from its position in FIG. 3A to its position in FIG. 3B. The indication of the movement of the gantry 324 may be an indication that the gantry 324 is currently rotating (or will rotate) from the upright position to the second position. The indication of movement may include one or more movement parameters that define the movement of the gantry 324. Such movement parameters may include, but are not limited to, a speed of rotation of the movement of the gantry, a direction of rotation of the movement of the gantry, an acceleration of the movement of the gantry, and/or an angle of rotation of the movement of the gantry.

The speed of rotation of the gantry may refer to how fast the gantry 324 is spinning or rotating around a central axis (e.g., the center axis 325). The speed of rotation may be measured in terms of angular velocity of the gantry 324, quantifying the angle turned per unit of time, such as radians per second, degrees per second, or revolutions per minute (RPM). The speed of rotation of the gantry 324 may depend on its mass distribution, the forces acting on it, and the energy imparted by the radiotherapy machine 341 to initiate or sustain the rotation. Such parameters may be used by the analytics server in determining the speed of rotating of the gantry.

The direction of rotation of the gantry 324 may refer to a specific path or orientation in which the gantry 324 is rotating around the central axis (e.g., the center axis 325). The direction of rotation can be clockwise or counterclockwise when viewed from a particular reference point (such as from the couch 322). This parameter may depend on the mechanical configuration of the gantry system, the applied forces, and the intended operation of the gantry 324. Such parameters may be analyzed by the analytics server to determine the direction of rotation of the gantry 324.

The acceleration of the movement of the gantry 324 may refer to the rate at which the speed or velocity of the gantry's rotation or linear movement changes over time. Acceleration can be expressed in units such as meters per second squared (m/s²) or radians per second squared for angular motion. The acceleration of the movement may depend on the torque applied, the resistance encountered, and the inertia of the gantry 324. The analytics server may use these parameters to monitor changes in the motion dynamics of the radiotherapy machine 341 and determine the acceleration of the movement of the gantry 324.

The angle of rotation of the movement of the gantry 324 may refer to the total angular displacement the gantry 324 achieves as it rotates around a central axis (e.g., the center axis 325). This angle may be measured in degrees, radians, or revolutions, and can indicate how far the gantry has rotated from its initial position. In some embodiments, the upright position of the gantry 324, as shown in FIG. 3A, may be considered the initial position and measured at 0°, 0 rad, etc. The angle of rotation may depend on the direction of rotation, duration of rotation, the acceleration of the rotation, the applied torque, and/or the stopping forces acting on the gantry 324. Such parameters may be utilized by the analytics server to determine the rotational progress of the gantry 324 during movement and, thereby, determine a total acceleration or deceleration of the gantry 324.

The analytics server may use one or more of the movement parameters above to calculate one or more corresponding icon movement parameters to use in dynamically adjusting, moving, and/or relocating (e.g., rotating) the second subset of icons 308 relative to the display screen 326. In other words, as the display screen 326 rotates with the gantry 324 in accordance with the one or more movement parameters, the analytics server may determine corresponding icon movement parameters to apply to the second subset of icons 308, such that the second subset of icons 308 appear to maintain a first orientation (e.g., upright), as shown in FIGS. 3A-3D.

In some embodiments, the icon movement parameters are opposite the movement parameters of the gantry 324. For example, as the gantry 324 rotates counter-clockwise at an angle of rotation (e.g., approximately 45° counter-clockwise, as shown by the movement of the gantry 324 between FIGS. 3A-3B), the second subset of icons 308 may be relocated/adjusted in a clockwise rotation within the first position 302 about a secondary datum point 312 at an opposite angle of rotation (e.g., approximately 45° clockwise) to “cancel out” the opposite rotation of the gantry 324 and thereby maintain the upright orientation while the secondary datum point 312 (and by extension, the second subset of icons 308) rotates about a center datum point 314/center axis 325. The second subset of icons 308 may rotate about the secondary datum point 312, which may be associated with (e.g., defining or defined by) the first position 302. For example, the secondary datum point 312 may be in a center of the first position 302. In some embodiments, the secondary datum point 312 is a center point of the second subset of icons 308 and/or the first position 302. In some embodiments, the gantry 324 (and by extension, the display screen 326) rotates about the center axis 325, which may extend from the center datum point 314. As the gantry 324 (and by extension, the display screen 326) rotates about the center axis 325, the secondary datum point 312 may rotate about the center datum point 314. In this way, as the second subset of icons 308 rotates about the secondary datum point 312 consistent with the determined icon movement parameters, the second subset of icons 308 are relocated/adjusted/rotated at an angle corresponding to the angle of rotation of the gantry 324 but in the opposite direction, such that they appear to be rotating consistent with the movement of the gantry 324 while maintaining an orientation, such as an upright orientation. While the second subset of icons 308 is shown rotating clockwise to counteract the counterclockwise rotation of the gantry 324, it is understood that in embodiments in which the rotation of the gantry 324 is clockwise, the second subset of icons 308 may rotate counterclockwise to counteract the clockwise rotation of the gantry 324. In like manner, the second subset of icons 308 appear to rotate consistent with the movement of the gantry 324. As described herein, in either implementation, the second subset of icons 308 rotates in the direction opposite the gantry 324, thus appearing to rotate consistent with the movement of the gantry 324.

In some embodiments, the gantry 324 is able to laterally adjust position. In such embodiments, the movement of the gantry 324 is not confined to rotation. In such embodiments, the analytics server may determine one or more additional movement parameters, such as lateral direction, speed, position, etc. The analytics server may determine icon movement parameters corresponding to the lateral movement to apply to the second subset of icons 308 to adjust a position of the second subset of icons 308 during movement of the gantry 324, such that the second subset of icons 308 appear to not be moving laterally with the movement of the gantry 324. Upon determining the icon movement parameters, the analytics server may transmit corresponding control signals to the display screen to adjust a display of the second subset of icons 308 in accordance with the determined icon movement parameters.

Additionally, or alternatively to the second subset of icons 308, the display screen 326 may have displayed thereon the subset of icons 310. The subset of icons 310 may be positioned circumferentially about the center datum point 314, such as shown in FIGS. 3A-3D. The analytics server may determine additional or alternative icon movement parameters corresponding to the movement parameters of the gantry 324 to apply to the subset of icons 310, such that the subset of icons 310 appears to not be rotating relative to the movement of the gantry 324. For example, without instructions to relocate the subset of icons 310, during rotation of the gantry 324 (and by extension the display screen 326) the subset of icons 310 would be become illegible because they would no longer be in the upright orientation. Accordingly, in some embodiments, the analytics server determines one or icon movement parameters to apply to the subset of icons 310 to maintain an upright orientation of the subset of icons 310 regardless of the position of the display screen 326. As shown by the subset of icons 310 between FIGS. 3A-3D, the subset of icons 310 maintains a consistent orientation and position relative to a reference (e.g., the couch 322, gravity, etc.), while the display screen 326 is rotated about the center datum point 314. In this way, the subset of icons 310 appears not to be moving relative to the couch 322. While the subset of icons 310 is shown rotating clockwise to counteract the counterclockwise rotation of the gantry 324, it is understood that in embodiments in which the rotation of the gantry 324 is clockwise, the subset of icons 310 may rotate counterclockwise to counteract the clockwise rotation of the gantry 324. In such manner, the subset of icons 310 appears to be stationary with respect to the gantry 324. As described herein, in either implementation, the subset of icons 310 rotates in the direction opposite the gantry 324, thus appearing to be stationary relative to the gantry 324.

In some embodiments, the analytics server periodically (e.g., 1 ms, 60 hertz, etc.) relocates the second subset of icons 308 and/or the subset of icons 310 to continually update the orientation and/or position of the second subset of icons 308 and/or the subset of icons 310 consistent with the movement of the gantry 324.

In some embodiments, during rotation of the gantry 324, the second subset of icons 308 and/or the subset of icons 310 are maintained within bounded, respective areas. For example, the second subset of icons 308 may be positioned and maintained within the first position 302 during relocating of the second subset of icons 308. Additionally, or alternatively, the subset of icons 310 may be positioned and maintained within the area 304 during relocation of the subset of icons 310. Due to being maintained within the first position 302, the second subset of icons 308 may, in some embodiments, be positioned in an area not visible to a user of the gantry 324. For example, upon a continuation of rotation from FIG. 3B, the second subset of icons 308 (while being maintained in the first position 302) may be located behind the head of the patient 306 and then below the couch 322, when viewed from the vantage point illustrated in FIG. 3B. It may be desired to have the information displayed in the second subset of icons 308 visible and legible at all positions of the gantry 324. As such, in some embodiments, the analytics server may remove the second subset of icons 308 from the first position 302 upon receiving an indication that the gantry 324 has rotated or moved beyond a first defined position. In at least one embodiment, the first defined position may be correlated to a rotational position (e.g., a number of angles) of the gantry 324 relative to the upright position (as shown in FIG. 3A). In at least one embodiment, the gantry 324 may be in the first defined position in FIG. 3B. As the gantry 324 continues a counterclockwise rotational movement (as shown in FIG. 3C), the analytics server may transmit instructions to the display screen 326 to remove second subset of icons 308 from the first position 302 and display them at a second position 303 not obfuscated from view. In this way, the information presented by the second subset of icons 308 is not hidden from view beneath the couch 322 during rotation of the gantry 324 and remains visible to a user. By maintaining visibility of the second subset of icons 308 during setup of the gantry 324 in preparation for administration of the radiotherapy, the user can quickly and easily view all relevant data of the treatment regardless of the position of the gantry 324, thus reducing setup time and the overall time of administration of the radiotherapy, resulting in quicker radiotherapies.

The indication that the gantry 324 has rotated beyond the first defined position may be generated and transmitted by various methods. For example, the gantry 324 may include one or more actuators for rotating the gantry 324. The actuators may include feedback circuits to provide an indication of the position of the gantry 324. In such embodiments, the feedback loop may provide a signal to the analytics server once the position has reached or passed the first defined position. The first defined position may be set by a user through a user interface, such as on a pendant (e.g., the pendant 142 of FIG. 1) and/or another electronic device (e.g., the system administrator computer 150 of FIG. 1). In some embodiments, the gantry 324 may interact with one or more sensors, such as a limit switch that provides an electronic signal to the analytics server once a limit (e.g., the first defined position) is reached.

FIG. 3C illustrates the second subset of icons 308 being displayed at/in the second position 303 upon the gantry 324 reaching the first defined position. As the gantry 324 continues to rotate counter-clockwise, as shown in the progression of the gantry 324 in FIGS. 3C-3D, the second subset of icons 308 continues to rotate in the second position 303 about a tertiary datum point 316 within the second position 303 such that the second subset of icons 308 maintains a static orientation (e.g., upright) as the tertiary datum point 316 orbits the center datum point 314 during rotation of the gantry 324 (and by extension the display screen 326), thereby maintaining legibility.

In some embodiments, as the second subset of icons 308 is removed from the first position 302 and redisplayed at the second position 303, the subset of icons 310 are maintained in a static orientation as well, by rotating each of the icons of the subset of icons 310 about the center datum point 314 as the gantry 324 rotates about the center axis 325. The analytics server may transmit instructions to adjust a position of each pixel of each icon of the second subset of icons 308 and/or the subset of icons 310 to maintain the static orientation during rotation of the display screen 326. The analytics server may take into account one or more positional parameters such as a distance from the center datum point 314, a distance from the secondary datum point 312, a distance from the tertiary datum point 316, a speed of rotation of the gantry 324, an acceleration of the gantry 324, etc.

FIG. 3D illustrates the gantry 324 further rotating about the center axis 325 and the second subset of icons 308 and the subset of icons 310 further being adjusted in position/orientation, such that they maintain a static orientation, the second subset of icons 308 appearing to rotate consistent with the movement of the gantry 324 and/or the subset of icons 310 appearing to not be rotating relative to the movement of the gantry 324.

Turning now to FIGS. 4A-4D, a front view of a display screen 426 (which may be substantially similar to the display screen 226 of FIG. 2A) coupled (e.g., mounted, attached, fastened, adhered) to a gantry (e.g., the gantry 224 of FIGS. 2A-3D). As shown in FIG. 4A, a first position 402 (which may be substantially similar to the first position 302 of FIG. 3A) of the display screen 426 is shown visible, and a second position 403 (which may be substantially similar to the second position 303 of FIG. 3C) is hidden from view (shown as dashed lines) beneath/behind a couch 422 (which may be substantially similar to the couch 222 of FIG. 2A) when the display screen 426 is in a first orientation (e.g., upright). As the display screen rotates counterclockwise about a center datum point 414 (which may be substantially similar to the center datum point 314 of FIGS. 3B and 3D) to a second orientation (as shown in FIG. 4B), the first position 402 moves with a secondary datum point 412 (which may be substantially similar to the secondary datum point 312 of FIG. 3B) and remains visible, while the second position 403 moves with a tertiary datum point 416 (which may be substantially similar to the tertiary datum point 316 of FIGS. 3C-3D) and emerges from behind/beneath the occlusion of the couch 422. In continuing the rotation (as shown in FIG. 4C) the display screen 426 may reach a first defined position at which point, in response to reaching the first defined position, the analytics server may obfuscate and/or remove a second subset of icons 408 (which may be substantially similar to the second subset of icons 308 of FIG. 3A) from being presented at the first position 402 on the display. The radiotherapy machine may include, in some embodiments, at least one position sensor, rotary encoder, or software-based positional tracking to detect when the gantry reaches the first defined position or a corresponding predefined angle. Upon reaching the first defined position, the analytics server may execute a GUI update (e.g., refresh, reload, add, and/or replace some or all of the GUI) by removing, dimming, greying out, or otherwise obfuscating certain icons (e.g., the second subset of icons 408). This process can be implemented through software-driven GUI rendering techniques, such as adjusting transparency levels, altering contrast, or replacing specific icons with placeholders.

In some implementations, the system may allow configurable obfuscation rules, enabling technicians, users, and/or administrators to define which icons should be obfuscated, hidden, dimmed, and/or replaced at specific gantry positions. Additionally, obfuscation may be time-dependent, where the second subset of icons 408 gradually fade out from the first position 402 after a brief delay.

Upon removing or otherwise obfuscating the second subset of icons 408 from display at the first position 402, the analytics server may present for display the second subset of icons 408 at the second position 403. In some embodiments, the analytics server may transmit one or more control signals to the display screen 426 to present for display the second subset of icons 408 at the second position 403. FIG. 4D illustrates the display screen 426 further rotated counterclockwise farther past the first defined position shown in FIG. 4C. In some embodiments, the analytics server may transmit instructions to disable the display screen 426 upon the gantry reaching a defined location (e.g., a radiotherapy position used in administering the radiotherapy). Disabling the display screen may include removing power to the display screen 426.

As described above, the radiotherapy machine may include position sensors, encoders, or feedback circuits integrated into the gantry's movement mechanism, allowing the system's processor to detect when the gantry reaches the defined location—such as a treatment position. Upon detecting that the gantry has reached the defined location, the analytics server transmits a control signal to disable the display screen 426, either by powering it down, dimming the screen, or entering a low-power standby mode. The system may also store configurable parameters, allowing users to set or adjust the defined position for screen disabling based on treatment protocols, system setup, user preferences, or safety requirements.

In addition or alternative to fully powering down (e.g., disabling) the display screen when the gantry reaches the defined location, the system may implement several alternative methods. For example, an alternative approach involves dimming the screen to a lower brightness level or activating a dark mode. Another alternative may include entering a standby mode, in which the screen remains active but ceases to display specific elements, instead showing, for example, a minimal indicator confirming that the system is operational.

In some embodiments, the system initiates a context-based reconfiguration upon the gantry reaching the defined location, where the display automatically shifts to a different interface mode when the gantry reaches the defined position. Instead of disabling the display screen 426, the GUI could transition to a simple status display with essential metrics such as treatment progress, patient positioning confirmation, or real-time safety alerts.

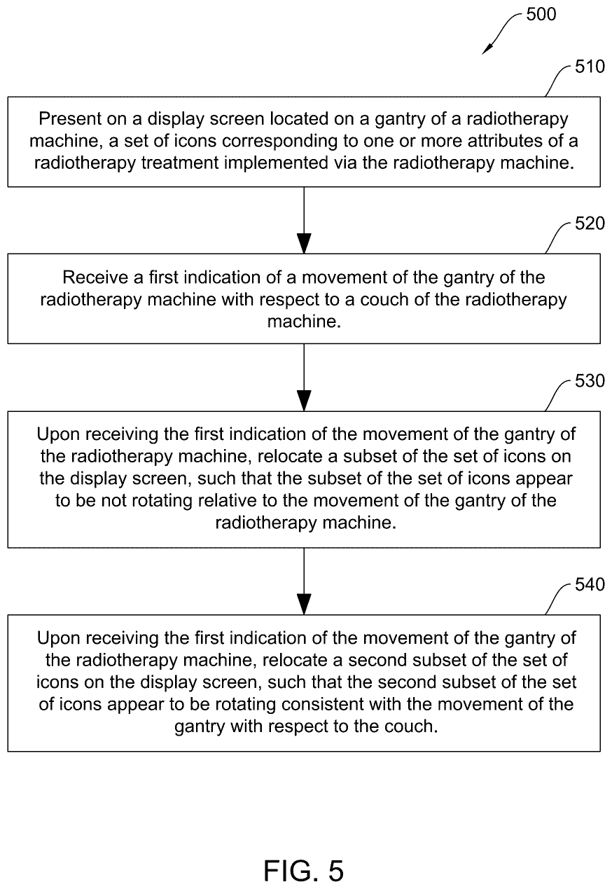

FIG. 5 is a flowchart of an example method 500 for dynamically adjusting orientation and position of one or more icons on a display screen coupled to a radiotherapy machine. The method 500 may be executed by one or more processors of the one or more electronic described herein, such as the analytics server 110, the pendant 142, the system administrator computer 150, the radiotherapy system 140, the radiotherapy machine 141, etc. The one or more processors may execute instructions stored on computer-readable, non-transitory medium, which may cause the one or more processors to execute steps comprising the steps 510, 520, 530, and/or 540. While steps 510, 520, 530, and 540 are shown in a specific order, it should be understood that the method 500 may comprise more, less, and/or different steps than those depicted in FIG. 5. Likewise, the order of the steps 510, 520, 530, and 540 are shown in FIG. 5 for illustrative purposes. However, it is understood that the steps of method 500 may be performed in any number of configurations or orders without departing from the scope of the systems and methods described herein. By way of example, the method 500 may include only steps 510, 520, and 530. In some embodiments, the method 500 may include only steps 510, 520, and 540. In some embodiments, the method 500 may include steps 510, 520, 530, and 540.

At step 510, one or more processors may present on a display screen located on a gantry of a radiotherapy machine, a set of icons corresponding to one or more attributes of a radiotherapy treatment implemented via the radiotherapy machine. As described herein, the one or more processors may transmit instructions to a display to display a set of icons comprised of one or more subset of icons, such as the first and subset of icons of FIGS. 3A-4D. The icons may display information related to administration of a radiotherapy treatment to a patient. This data may include personal information of the patient and/or positioning data for a radiotherapy machine and/or a couch (e.g., the couch 222 of FIG. 2A) upon which the user is placed during administration of the radiotherapy treatment.

At step 520, one or more processors may receive a first indication of a movement of the gantry of the radiotherapy machine with respect to a couch of the radiotherapy machine. The gantry of the radiotherapy machine to which the display screen is coupled may be configured to move along or about one or more axes. By way of example, the gantry may be configured to rotate about a center axis (e.g., the center axis 225 of FIG. 2A) to position the radiotherapy machine for administration of the radiotherapy.

When the gantry is moved, or an instruction is provided to the gantry which instructs the gantry to move, the one or more processors may receive an indication of the movement and/or instructions. This indication may come from a controller which sends control signals to the gantry to move. In some embodiments, the one or more processors receive the indication of movement of the gantry from feedback loop of one or more actuators coupled to the gantry and configured to adjust the position of the gantry. These feedback loops may provide data packets to the one or more processors with movement parameters of the gantry, such as speed of rotation, angular acceleration, time of movement, etc.

At step 530, upon receiving the first indication of the movement of the gantry of the radiotherapy machine, one or more processors may relocate a subset of the set of icons on the display screen, such that the subset of the set of icons appear to be not rotating relative to the movement of the gantry of the radiotherapy machine. Upon receiving the indication of the movement of the gantry and the associated movement parameters of the gantry, the one or more processors may determine corresponding icon movement parameters to apply to the subset of the set of icons. In applying the determined corresponding icon movement parameters, the subset of icons are relocated on the display screen such that they maintain an orientation relative to a reference (e.g., the couch, gravity, the ground, etc.), despite the movement of the display screen, which is coupled to the gantry. The icon movement parameters may be used to counter the movement of the gantry, such that the subset of icons appear to be static with respect to a reference (e.g., the couch, gravity, the ground, etc.), even though the gantry (and by extension, the display screen) are rotating about an axis.

The one or more processors may determine control signals in accordance with the icon movement parameters and transmit the control signals to the display screen, such that the display screen displays the subset of icons in accordance with the icon movement parameters.

At step 540, upon receiving the first indication of the movement of the gantry of the radiotherapy machine, one or more processors may relocate a second subset of the set of icons on the display screen, such that the second subset of the set of icons appear to be rotating consistent with the movement of the gantry. Upon receiving the indication of the movement of the gantry and the associated movement parameters of the gantry, the one or more processors may determine corresponding icon movement parameters to apply to the second subset of the set of icons. In applying the determined corresponding icon movement parameters, the second subset of icons is relocated on the display screen, such that they maintain an orientation relative to a reference (e.g., the couch, gravity, the ground, etc.), despite the movement of the display screen, which is coupled to the gantry. The icon movement parameters may be used to counter the movement of the gantry, such that the second subset of icons appear to be rotating consistent with the movement of the gantry with respect to a reference (e.g., the couch, gravity, the ground, etc.).

The one or more processors may determine control signals in accordance with the icon movement parameters and transmit the control signals to the display screen, such that the display screen displays the second subset of icons in accordance with the icon movement parameters.

### Example Clauses

Further aspects of these teachings are provided by the subject matter of the following clauses.

Clause 1. A computer-readable medium with instructions stored therein that when executed by one or more processors, cause the one or more processors to: present on a display screen located on a gantry of a radiotherapy machine, a set of icons corresponding to one or more attributes of a radiotherapy treatment implemented via the radiotherapy machine; receive a first indication of a movement of the gantry of the radiotherapy machine; and upon receiving the first indication of the movement of the gantry of the radiotherapy machine, relocate a subset of the set of icons on the display screen, such that the subset of the set of icons appears to be not rotating relative to a movement of the gantry of the radiotherapy machine.

Clause 2. The computer-readable medium of clause 1, wherein the instructions further cause the one or more processors to, upon receiving the first indication of the movement of the gantry of the radiotherapy machine, relocate a second subset of the set of icons on the display screen, such that the second subset of the set of icons appear to be rotating consistent with the movement of the gantry.

Clause 3. The computer-readable medium of clauses 1-2, wherein the instructions further cause the one or more processors to: remove the second subset of the set of icons from a first position when the gantry has rotated beyond a first defined position; and display at a second position the second subset of the set of icons previously removed when the gantry has rotated beyond a second defined position.

Clause 4. The computer-readable medium of clauses 1-3, wherein the second subset of the set of icons displays an identifier of a patient being treated.

Clause 5. The computer-readable medium of clauses 1-4, wherein the instructions further cause the one or more processors to: disable the display screen when the gantry is at a defined position.

Clause 6. The computer-readable medium of clauses 1-5, wherein the instructions further cause the one or more processors to: obfuscate at least one icon within the set of icons when the gantry reaches a defined position.

Clause 7. The computer-readable medium of clauses 2-6, wherein relocating of the second subset of the set of icons is consistent with a movement parameter of the movement of the gantry of the radiotherapy machine.

Clause 8. The computer-readable medium of clauses 1-7, wherein the instructions further cause the one or more processors to: receive a second indication of the movement parameter of the gantry of the radiotherapy machine; determine an icon movement parameter for the second subset of the set of icons, wherein the icon movement parameter corresponds to the movement parameter of the gantry; and upon receiving the first indication of the movement of the gantry of the radiotherapy machine, relocate the second subset of the set of icons on the display screen in accordance with the icon movement parameter, such that the second subset of the set of icons appear to be rotating with the movement of the gantry.

Clause 9. The computer-readable medium of clauses 1-8, wherein the movement parameter of the gantry of the radiotherapy machine comprises at least one of a speed of rotation of the movement of the gantry, a direction of rotation of the movement of the gantry, an acceleration of the movement of the gantry, or an angle of rotation of the movement of the gantry.

Clause 10. The computer-readable medium of clauses 1-9, wherein relocating the second subset of the set of icons on the display screen, such that the second subset of the set of icons appear to be rotating consistent with the movement of the gantry further comprises: adjusting the second subset of the set of icons with respect to the display screen at a first angle of rotation opposite of a second angle of rotation of the movement of the gantry.

Clause 11. The computer-readable medium of clauses 1-10, wherein the instructions further cause the one or more processors to: maintain a location of the second subset of the set of icons relative to the display screen such that the second subset of the set of icons rotate about a datum point on the display screen and wherein the second subset of the set of icons are further adjusted at the first angle of rotation about a secondary datum point.

Clause 12. A system comprising: a radiotherapy machine comprising a gantry with a display screen located thereon; one or more processors; and a computer-readable, non-transitory medium with instructions stored therein that when executed by the one or more processors, cause the one or more processors to: present on the display screen located on the gantry of the radiotherapy machine, a set of icons corresponding to one or more attributes of a radiotherapy treatment implemented via the radiotherapy machine; receive a first indication of a movement of the gantry of the radiotherapy machine; and upon receiving the first indication of the movement of the gantry of the radiotherapy machine, relocate a subset of the set of icons on the display screen, such that the subset of the set of icons appear to be not rotating relative to the movement of the gantry of the radiotherapy machine.

Clause 13. The system of clause 12, wherein the instructions further cause the one or more processors to, upon receiving the first indication of the movement of the gantry of the radiotherapy machine, relocate a second subset of the set of icons on the display screen, such that the second subset of the set of icons appear to be rotating consistent with the movement of the gantry.

Clause 14. The system of clauses 12-13, wherein the instructions further cause the one or more processors to: remove the second subset of the set of icons from a first position when the gantry has rotated beyond a first defined position; and display at a second position the second subset of the set of icons previously removed when the gantry has rotated beyond a second defined position.

Clause 15. The system of clauses 12-14, wherein relocating of the second subset of the set of icons corresponds with a movement parameter of the movement of the gantry of the radiotherapy machine.

Clause 16. The system of clauses 12-15, wherein the instructions further cause the one or more processors to: receive a second indication of the movement parameter of the gantry of the radiotherapy machine; determine an icon movement parameter for the second subset of the set of icons, wherein the icon movement parameter corresponds to the movement parameter of the gantry; and upon receiving the first indication of the movement of the gantry of the radiotherapy machine, relocate the second subset of the set of icons on the display screen in accordance with the icon movement parameter, such that the second subset of the set of icons appear to be rotating with the movement of the gantry while maintaining a first orientation.

Clause 17. The system of clauses 12-16, wherein the instructions further cause the one or more processors to adjust a first orientation of the second subset of the set of icons with respect to the display screen at a first angle of rotation opposite of a second angle of rotation of the movement of the gantry such that a second orientation of the second subset of the set of icons is maintained when relocating the second subset of the set of icons on the display screen, such that the second subset of the set of icons appears to be rotating consistent with the movement of the gantry.

Clause 18. The system of clauses 12-17, wherein the instructions further cause the one or more processors to maintain a location of the second subset of the set of icons relative to the display screen such that the second subset of the set of icons rotate about a datum point on the display screen.

Clause 19. A method comprising: presenting, by at least one processor on a display screen located on a gantry of a radiotherapy machine, a set of icons corresponding to one or more attributes of a radiotherapy treatment implemented via the radiotherapy machine; receiving, by the at least one processor, a first indication of a movement of the gantry of the radiotherapy machine; and upon receiving the first indication of the movement of the gantry of the radiotherapy machine, relocating, by the at least one processor, a subset of the set of icons on the display screen, such that the subset of the set of icons appear to be not rotating relative to the movement of the gantry of the radiotherapy machine.

Clause 20. The method of clause 19, further comprising: upon receiving the first indication of the movement of the gantry of the radiotherapy machine, relocating, by the at least one processor, a second subset of the set of icons on the display screen, such that the second subset of the set of icons appear to be rotating consistent with the movement of the gantry.

Clause 21: A computer program comprising instructions which, when the program is executed by a computer, cause the computer to carry out the method of any of clauses 19-20.

Clause 22: A computer-readable medium comprising a set of instructions, that when executed by one or more processors, cause the one or more processors to carry out the method of any of clauses 19-20.

The foregoing method descriptions and the process flow diagrams are provided merely as illustrative examples and are not intended to require or imply that the steps of the various embodiments must be performed in the order presented. The steps in the foregoing embodiments may be performed in any order. Words such as “then,” “next,” etc. are not intended to limit the order of the steps; these words are simply used to guide the reader through the description of the methods. Although process flow diagrams may describe the operations as a sequential process, many of the operations can be performed in parallel or concurrently. In addition, the order of the operations may be re-arranged. A process may correspond to a method, a function, a procedure, a subroutine, a subprogram, and the like. When a process corresponds to a function, the process termination may correspond to a return of the function to a calling function or a main function.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

The actual software code or specialized control hardware used to implement these systems and methods is not limiting of the invention. Thus, the operation and behavior of the systems and methods were described without reference to the specific software code being understood that software and control hardware can be designed to implement the systems and methods based on the description herein.

When implemented in software, the functions may be stored therein as one or more instructions or code on a computer-readable, non-transitory medium or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM, or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting with the true scope and spirit being indicated by the following claims.

What we claim is:

1. A computer-readable medium comprising instructions, that when executed by one or more processors, cause the one or more processors to:
present on a display screen located on a gantry of a radiotherapy machine, a set of icons corresponding to one or more attributes of a radiotherapy treatment implemented via the radiotherapy machine;
receive a first indication of a movement of the gantry of the radiotherapy machine; and
upon receiving the first indication of the movement of the gantry of the radiotherapy machine, relocate a subset of the set of icons on the display screen, such that the subset of the set of icons appears to be not rotating relative to a movement of the gantry of the radiotherapy machine.

2. The computer-readable medium of claim 1, wherein the instructions further cause the one or more processors to, upon receiving the first indication of the movement of the gantry of the radiotherapy machine, relocate a second subset of the set of icons on the display screen, such that the second subset of the set of icons appear to be rotating consistent with the movement of the gantry.

3. The computer-readable medium of claim 2, wherein the instructions further cause the one or more processors to:
remove the second subset of the set of icons from a first position when the gantry has rotated beyond a first defined position; and
display at a second position the second subset of the set of icons previously removed when the gantry has rotated beyond a second defined position.

4. The computer-readable medium of claim 2, wherein the second subset of the set of icons displays an identifier of a patient being treated.

5. The computer-readable medium of claim 1, wherein the instructions further cause the one or more processors to:
disabling the display screen when the gantry is at a defined position.

6. The computer-readable medium of claim 1, wherein the instructions further cause the one or more processors to:
obfuscate at least one icon within the set of icons when the gantry reaches a defined position.

7. The computer-readable medium of claim 2, wherein relocating of the second subset of the set of icons is consistent with a movement parameter of the movement of the gantry of the radiotherapy machine.

8. The computer-readable medium of claim 7, wherein the instructions further cause the one or more processors to:
receive a second indication of the movement parameter of the gantry of the radiotherapy machine;
determine an icon movement parameter for the second subset of the set of icons, wherein the icon movement parameter corresponds to the movement parameter of the gantry; and
upon receiving the first indication of the movement of the gantry of the radiotherapy machine, relocate the second subset of the set of icons on the display screen in accordance with the icon movement parameter, such that the second subset of the set of icons appear to be rotating with the movement of the gantry.

9. The computer-readable medium of claim 8, wherein the movement parameter of the gantry of the radiotherapy machine comprises at least one of a speed of rotation of the movement of the gantry, a direction of rotation of the movement of the gantry, an acceleration of the movement of the gantry, or an angle of rotation of the movement of the gantry.

10. The computer-readable medium of claim 2, wherein relocating the second subset of the set of icons on the display screen, such that the second subset of the set of icons appear to be rotating consistent with the movement of the gantry further comprises:
adjusting the second subset of the set of icons with respect to the display screen at a first angle of rotation opposite of a second angle of rotation of the movement of the gantry.

11. The computer-readable medium of claim 10, wherein the instructions further cause the one or more processors to:
maintain a location of the second subset of the set of icons relative to the display screen such that the second subset of the set of icons rotate about a datum point on the display screen and wherein the second subset of the set of icons are further adjusted at the first angle of rotation about a secondary datum point.

12. A system comprising:
a radiotherapy machine comprising a gantry with a display screen located thereon;
one or more processors; and
a computer-readable, non-transitory medium with instructions stored therein that when executed by the one or more processors, cause the one or more processors to:
present on the display screen located on the gantry of the radiotherapy machine, a set of icons corresponding to one or more attributes of a radiotherapy treatment implemented via the radiotherapy machine;
receive a first indication of a movement of the gantry of the radiotherapy machine; and
upon receiving the first indication of the movement of the gantry of the radiotherapy machine, relocate a subset of the set of icons on the display screen, such that the subset of the set of icons appear to be not rotating relative to the movement of the gantry of the radiotherapy machine.

13. The system of claim 12, wherein the instructions further cause the one or more processors to, upon receiving the first indication of the movement of the gantry of the radiotherapy machine, relocate a second subset of the set of icons on the display screen, such that the second subset of the set of icons appear to be rotating consistent with the movement of the gantry.

14. The system of claim 13, wherein the instructions further cause the one or more processors to:
remove the second subset of the set of icons from a first position when the gantry has rotated beyond a first defined position; and
display at a second position the second subset of the set of icons previously removed when the gantry has rotated beyond a second defined position.

15. The system of claim 13, wherein relocating of the second subset of the set of icons corresponds with a movement parameter of the movement of the gantry of the radiotherapy machine.

16. The system of claim 15, wherein the instructions further cause the one or more processors to:
receive a second indication of the movement parameter of the gantry of the radiotherapy machine;
determine an icon movement parameter for the second subset of the set of icons, wherein the icon movement parameter corresponds to the movement parameter of the gantry; and
upon receiving the first indication of the movement of the gantry of the radiotherapy machine, relocate the second subset of the set of icons on the display screen in accordance with the icon movement parameter, such that the second subset of the set of icons appear to be rotating with the movement of the gantry while maintaining a first orientation.

17. The system of claim 13, wherein the instructions further cause the one or more processors to adjust a first orientation of the second subset of the set of icons with respect to the display screen at a first angle of rotation opposite of a second angle of rotation of the movement of the gantry such that a second orientation of the second subset of the set of icons is maintained when relocating the second subset of the set of icons on the display screen, such that the second subset of the set of icons appears to be rotating consistent with the movement of the gantry.

18. The system of claim 17, wherein the instructions further cause the one or more processors to maintain a location of the second subset of the set of icons relative to the display screen such that the second subset of the set of icons rotate about a datum point on the display screen.

19. A method comprising:
presenting, by at least one processor on a display screen located on a gantry of a radiotherapy machine, a set of icons corresponding to one or more attributes of a radiotherapy treatment implemented via the radiotherapy machine;
receiving, by the at least one processor, a first indication of a movement of the gantry of the radiotherapy machine; and
upon receiving the first indication of the movement of the gantry of the radiotherapy machine, relocating, by the at least one processor, a subset of the set of icons on the display screen, such that the subset of the set of icons appear to be not rotating relative to the movement of the gantry of the radiotherapy machine.

20. The method of claim 19, further comprising:
upon receiving the first indication of the movement of the gantry of the radiotherapy machine, relocating, by the at least one processor, a second subset of the set of icons on the display screen, such that the second subset of the set of icons appear to be rotating consistent with the movement of the gantry.
